(19) United States
(12) Patent Application Publication
(10) Pub. No.: US 2006/0235360 A1
(43) Pub. Date: Oct. 19, 2006

(54) Water-storage material

(75) Inventors: Michael Ahlers, Eberbach (DE); Brigitte Fischer, Konigswinter (DE)

Correspondence Address: LEYDIG VOIT & MAYER, LTD, 700 THIRTEENTH ST. NW, SUITE 300, WASHINGTON, DC, 20005-3960, US

(73) Assignee: Gelita AG, Eberbach (DE)

(21) Appl. No.: 11/350,901

(22) Filed: Feb. 10, 2006

Related U.S. Application Data
(63) Continuation of application No. PCT/EP04/08762, filed on Aug. 5, 2004.

(30) Foreign Application Priority Data
Aug. 21, 2003 (DE) 103 39 178.9

(51) Int. Cl. A61F 13/15

(52) U.S. Cl. 604/365

(57) ABSTRACT

A water-storage material comprising a flat biodegradable carrier material and a water absorber, the particles of the water absorber being fixed in position on the carrier material in an evenly distributed fashion, is disclosed.

### CROSS REFERENCE TO RELATED APPLICATIONS

This application is a continuation of international application number PCT/EP2004/008762 filed on Aug. 5, 2004, that claims the benefit of German application number 103 39 178.9, filed on Aug. 21, 2003, both which are incorporated herein by reference in their entirety and for all purposes.

### BACKGROUND OF THE INVENTION

The invention relates to a water-storage material for providing plants with a long-term supply of water.

DE 44 27 292 A1 proposes the insertion of natural sponge into the root zone of the plants so as to provide a reservoir which improves initial rooting and can save watering operations. However, the water-storage effect achieved thereby is very limited.

The same applies to the water-storage compositions composed mainly of compost, such as are described in DE 1 99 20 641 A1. In particular, when these materials are placed in the root zone of plants, the pressure then bearing on the water-storage material causes them to lose a considerable part of their water-storing capacity, which is from the outset limited. Furthermore, such materials are not infrequently pollutant-laden by reason of the fact that the origin of their main component, compost, cannot be discerned with certainty.

So-called superabsorbers based on polyacrylates, polyacrylamides, polyvinyl amines, and derivatives thereof, which can form gels when they absorb water and take up many times their own weight of water, are highly suitable water absorbers. This is due, on the one hand, to the aforementioned high water-absorbing capacity, and, on the other hand, to the fact that the stored water is retained by the absorber particles and is not released even under high pressure. This is accompanied by the property of the continued capability of taking up water even when subjected to high applied pressure.

When excess liquid is present, these particulate, usually granular water absorbers take up and bind the liquid and are capable of releasing the stored water to the environment as it gets drier.

One of the producers of so-called superabsorbers (eg Luquasorb®, sold by BASF AG) recommends that the granules supplied in large packs be worked into the soil at a rate of from 2 to 50 g/m². This is intended to increase the resistance potential of the plants to insufficient irrigation and dryness to a considerable extent and to greatly reduce the number of necessary watering operations with consequently reduced water consumption.

Further advantages of working the superabsorber granules into the soil are described as being the loosening of the soil as caused by the expansion and shrinkage of the particles of granular material as they absorb and release water respectively. An additional effect is described as being the improved aeration of the roots.

Although these effects of superabsorbers are convincing, the superabsorbers are not, in fact, easy to use in practice.In particular, gardeners experience dosage problems when handling the granules. Local overdosage can cause the plants to lift or, at the worst, to tip over, this being due to the large increase in volume of the particles of superabsorber granular material arising when they absorb water. Furthermore, agglomerates of granules can form which reduce their water-storing capacity due to blocking of the particles.

Hitherto, this dosage problem has been counteracted by supplying the particulate superabsorber in bulk form in permeable bags of non-woven material for placement in the root zone of plants. However, these are at best only effective when used over small areas. Use thereof on lawns is thus not practicable.

### BRIEF SUMMARY OF THE INVENTION

It is an object of the present invention to provide a water-storage material which achieves maximum water-storing capacity, based on water absorber used, and which is simple to apply in correct dosage over small and large areas.

This object is achieved in the present invention in that the water-storage material defined above comprises a flat biodegradable carrier material and a particulate water absorber, the particles of water absorber being fixed on the carrier material in an evenly distributed fashion.

In the case of the water-storage material of the invention, fixation of the evenly distributed water absorber particles on the carrier material ensures that each particle is fully available for its water-storage function, and agglomeration of the particles is reliably avoided. The primary purpose of the carrier material is to keep the particles evenly distributed over a specific area.

Fixing of the particles can, for example, be achieved by applying a preferably likewise biodegradable adhesive to the carrier material and then scattering the water absorber particles thereon.

Alternatively, the carrier material may contain an adhesive agent, which is activated prior to application of the water absorber particles.

The adhesive agents used are preferably such as readily allow access of water to the particles, ie, the adhesive agents should not completely enclose the water absorber particles or should be at least sufficiently water-permeable for any water present to be immediately taken up by the water absorber particles. This is very simple to accomplish when the carrier material itself contains the adhesive agent.

The process of water absorption and subsequent release of the stored water to the dry environment can take place virtually as often as desired, particularly when the aforementioned so-called superabsorbers are used as the water absorbers.

Preferably, use is made of an adhesive agent that is swellable in water so that this in itself increases the water absorption and water-storing capacity of the water-storage material. This contribution to the water-storing capacity naturally declines with time on account of the required biodegradation of the adhesive agent.

The adhesive agents to be used in the present invention may well be water-soluble, since the adhesive agents have usually fulfilled their purpose once the water-storage material has been embedded in the soil in the root zone of the plants, for the particulate water absorbers are then kept in adequately spaced relationship by the soil itself.

In consideration of the above factors, suitable adhesive agents are polypeptides, particular preference being given to collagen hydrolysate.

In this case, in particular, collagen hydrolysate having an average molecular weight \overline{M}_{w} ranging from 1,000 to 20,000 dalton, particularly from 3,000 to 15,000 dalton, is preferred.

The carrier material itself is selected from biocompatible, especially biodegradable materials, particularly suitable materials being polymers such as polysaccharides, polypeptides, or polyvinyl alcohol.

In particular, biopolymers, such as collagens, gelatin, starch, or cellulose can be used. The above materials can be used alone or intermixed.

These polymers have the additional advantage that they, too, biodegrade with time so that the water-storage material of the invention can subsequently remain in the soil in its entirety, or can alternatively be composted with the ambient soil.

The carrier material used in the present invention is preferably in the form of flexible material such as can be wrapped, for example, around root balls of plants offered for sale, in order to keep the root balls moist over prolonged periods of time, for example during transport.

Preference is given to water-storage materials which show, even in the dry state, adequate flexibility and the ability to be shaped in any desired manner, for example when being wrapped around root balls. Therefore preferred carrier materials are flexible sheets and foam sheets.

If the biodegradable material of the carrier has no inherent flexibility, plasticizers can be added.

Recommended substances are, for example, low-molecular plasticizers such as lecithins, fats, fatty acids, glycerol, diglycerol, triglycerol, and sorbitols. Other recommended substances are particularly hygroscopic polymers, since water is frequently the best plasticizer for biopolymers, examples being polyglycerol, polyoxyalkylenes, polyoxyalkylene esters, polyvinyl alcohols, polyvinylpyrrolidone, PVFm, PVAm, sorbitan esters, and cellulose derivatives such as CMC.

More preferably, the carrier material exhibits its own water-storing capacity, ie, use is preferably made of expanded material, particularly open-pore expanded material.

It is recommended that the expanded material has a dry density ranging from 5 to 50 mg/cm³. On the one hand, such materials can be produced with a sufficiently high mechanical strength for handling purposes, whilst on the other hand the weight of the carrier material itself is minimal.

The carrier material can alternatively be in the form of a non-woven structure or network structure so that the enlarged surface area of the carrier material can allow for an increased number of particles of the water absorber to be bonded by the adhesive agent.

In another variant of the water-storage material of the invention, particulate fertilizer is evenly distributed over the carrier material in addition to the particles of water absorber. The same instructions apply to fixing of the fertilizer particles as those given above regarding fixing of the absorber particles.

Here again, the even distribution of the fertilizer particles on the carrier material has an advantageous effect, since this guarantees an evenly spread supply of fertilizing substances to the soil.

The water-storage materials of the invention are particularly suitable for use as water-storage basins for plants, particularly those planted in plant receptacles, where the water-storage material can thus ensure continuous moisture penetration of the soil. On account of the use of particulate water absorbers fixed in a distributed fashion on a carrier material according to the present invention, the full capacity of the water absorbers is available for the water-storage effect, since agglomeration of the water absorber particles cannot now occur. On the contrary, all of the particles are fully available for effecting water-storage. The same applies to fertilizer particles which may be similarly fixed on the carrier material in distributed fashion.

If certain structures of the carrier material are used, such as a coarse network structure, there is an increase in the surface area on which the water absorber particles can be bonded, optionally in conjunction with fertilizer particles.

The water-storage function of the carrier material itself presents yet another water-storage effect, and the water-storage materials that can be used are such as must not necessarily hold water under pressure, ie, they can be expanded materials, for example, particularly open-pore materials such as expanded polysaccharides, polypeptides, or PVA.

The carrier materials used can themselves contain an adhesive agent or may even themselves be capable of being activated as adhesive agents. Thus gelatin, for example, when used as carrier material, can be activated as adhesive agent simply by wetting with water so that the water absorber particles can be readily distributed over the flat carrier material and directly fixed in position thereon by the wetted gelatin.

Conceivably, a carrier material might be used which is a relatively coarsely porous, open-pore foam, on which the particles of water absorber are not only bonded to the macroscopic surface but are also enclosed in the open pores of the expanded structure, where they are fixed in position by the adhesive.

Particularly those water-storage materials which are to remain permanently in the soil will be such as allow breakthrough of roots.

### BRIEF DESCRIPTION OF THE SEVERAL VIEW OF THE DRAWINGS

These and other advantages of the invention are illustrated below in greater detail with reference to the drawings in which:

FIG. 1 is a perspective view of a water-storage material of the invention;

FIG. 2 is a detailed view of a border area of the material shown in FIG. 1; and

FIG. 3 shows another embodiment of the water-storage material of the invention.

### DETAILED DESCRIPTION OF THE INVENTION

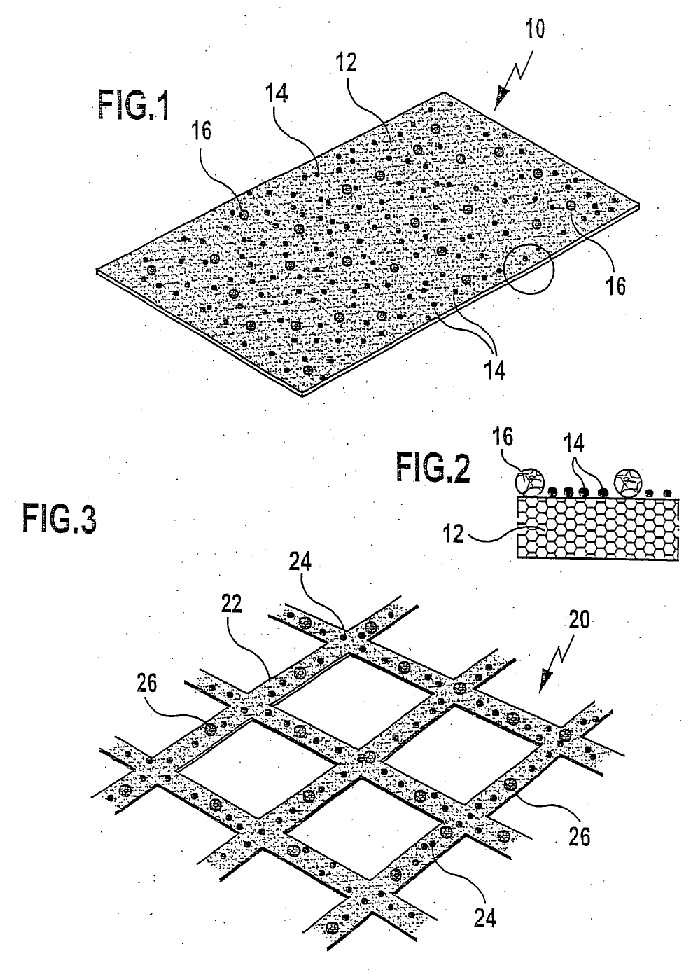

FIG. 1 shows a water-storage material denoted by reference numeral 10 and comprising a flat carrier material 12, in particular an expanded polypeptide, which contains, on the top surface thereof, a large number of evenly distributed particles 14 of a water absorber. In this case the particles of water absorber are bonded by an adhesive agent, in particular a collagen hydrolysate having an average molecular weight in the range of from 3,000 to 15,000 dalton. Fertilizer particles 16 may also be bonded thereto.

The carrier material 12 is, as shown in FIG. 2, an expanded polypeptide and thus has its own water-storage function acting in addition to the water-storing capacity of the particles 14 of superabsorber.

FIG. 3 shows another variant of the water-storage material of the invention, designated in this case by reference numeral 20. The water-storage material 20 used here comprises a flat carrier material 22 in the form of a two-dimensional net, in which the network structure may comprise rectangular meshes, or alternatively of course, meshes of any other desired topology.

Particles 24 of a superabsorber are bonded to the surface of the individual lands between the individual junctions of the network structure of the flat carrier material 22, the adhesive agent used again being preferably collagen hydrolysate having an average molecular weight of from 3,000 to 15,000 dalton.

The water-storage materials of the invention having a flat carrier material on which the water absorber particles are evenly distributed and bonded thereto have not only the advantage that the individual particles of the water absorber are all available with their full capacity and cannot agglomerate with each other, but said materials also have the advantage that the carrier material may itself be adapted such that it fulfills an additional water-storage function. In addition to the particles of absorber, fertilizer particles 16, 26 may be bonded to the flat carrier materials 12, 22 and thus be present thereon in an evenly distributed fashion.

If use is made of a carrier material 12, 22 of a biodegradable material, such as a polysaccharides, polypeptide, or PVA, the entire water-storage material can remain in the soil until it has fully disintegrated due to degradation, or it can be composted at any time together with the ambient soil. For such a case it is particularly recommended to use collagen hydrolysate as adhesive agent since this shows very little resistance to biodegradation in the soil.

The superabsorbers recommended as water absorbers, which are usually based on polyacrylic acid polymers, polyacrylamides, polyvinyl amines, and derivatives thereof, should show much greater resistance to biodegradation, in order to ensure that the long-term effect of the water-storage function extends beyond the time required for degradation of the carrier material. The aforementioned polymers are biocompatible and can therefore remain permanently in the soil.

The present invention is illustrated in greater detail below in a number of further aspects with reference to the following examples:

### EXAMPLES

### 1. Production of an Expanded Flat Carrier Material

A 15% solution of a pig rind gelatin having a high Bloom number is produced by preliminary swelling of the granules in cold water followed by dissolution at 60° C. The solution is then cooled to 45° C. and used for the production of a foam by means of continuous aeration (equipment: Mondomix Mini). Using a pumping rate of 450 rpm, a mixing head setting of 750 rpm and of a density setting of 75 mL there is obtained a moist density of 130 mg/cm³. The foam is extruded through a slot die into a box-shaped mold. This foamed cake is dried with conditioned air and then cut into panels measuring 20×20×0.6 cm. The specific density of the foam sheets is 25 mg/cm³.

Instead of using the relatively expensive, high-quality pig rind gelatin, no great loss of functionality occurs when gelatins of other quality and/or provenance, eg bone gelatin, are used.

Alternatively, it may be possible to blend the gelatin used to assist foam formation with substantially cheaper starch. Contents of starch of from 10 to 20 wt % or even higher do not hinder foam formation. On the contrary, expanded materials on a purely starch basis are possible, in which case a content of gelatin improves the stability of the carrier material.

In the case of a carrier material of collagen origin, the raw material is preferably selected such that from 10 to 55 wt % thereof, and preferably from 20 to 40 wt % thereof, has a molecular weight \overline{M}_{w} of <40,000 dalton. In this case, the adhesive action of the collagenic carrier material can be induced simply by contact with water. This applies, of course, to both expanded and non-expanded carrier materials.

Improvement of the Structural Integrity of the Carrier Material in the Hydrous State

In order to improve its dimensional stability, particularly in the hydrous state, the biodegradable expanded or non-expanded carrier material can be crosslinked during production or subsequently thereto. Any of the suitable crosslinking agents can be used for crosslinking. The biodegradability should be maintained in this case. For proteins, such as collagen hydrolysate, examples of suitable crosslinking agents are aldehydes, dialdehydes, diisocyanates, aliphatic and aromatic dihalogenides, reactive vinyl compounds, organic dicarboxylic acids in the form of active esters, and also inorganic crosslinking agents and complexers such as phosphates. Crosslinking is also successfully achieved by thermal dehydration and by UV irradiation. This also applies when the carrier material is in the form of a sheet of foam.

Depending on the degree of desired improvement in the mechanical properties, up to, say, 3,000 ppm of formaldehyde can be added (cf Example 3).

### 2. Production of a Water-Storage Material of the Invention

The carrier sheets or expanded sheets obtained in Example 1 are sprayed with water and, after a period of 10 min, during which the water-soluble components of collagen hydrolysate pass into the aqueous phase, a powder of an acrylate superabsorber is scattered thereover (1.25 g/100 cm²). After the absorber has become bonded to the carrier, the whole is dried. A foam sheet measuring 20×20×0.6 cm shows a water absorption of ca 1 L. Suitable superabsorbers are, for example, the materials sold by BASF AG under the trade name Luquasorb®.

Comparison of Water Absorption Capacities

| | Water absorption | | | |
|---|---|---|---|---|
| | per initial volume of the | | Water absorption | |
| Absorber | absorber used | | per mass | |
| Garden mold, moist | 0.4 | kg/L | 0.95 | kg/kg |
| Garden mold, dry | 1 | kg/L | 4 | kg/kg |
| Water-storage sheet of | 4.7 | kg/L | 140 | kg/kg |
| Example 2 | | | | |

Thus one of the sheets measuring 20×20×0.6 cm³ is fully adequate for, say, a boxwood plant having a height of from 50 to 100 cm. The introduction of a corresponding amount of bulk superabsorber particles (5 g) into the root zone of such a plant in an evenly distributed fashion is on the other hand in practice difficult to carry out and thus not infrequently prone to overdosing, as a result of which rooting of the plant, ie anchoring of the root system in the soil, is not assisted but, instead, hampered.

The above table demonstrates the superiority of the water-storage material of the invention over garden mold as regards the water-storing capacity thereof.

### 3. Production of a Further Water-Storage Material of the Invention

In a manner similar to that described in Example 1, a solution is prepared containing 13% of gelatin, 2% of diglycerol, 0.2% of dye (iron oxide brown) and 1,000 ppm of formaldehyde (based on gelatin). The solution is foamed as described and then extruded through a slot die onto a conveyor belt. A 6 mm thick gelatin foam is treated with superabsorber (1.25 g/1.25 cm²) and dried in a drying tunnel. The resulting product is flexible and can be deformed in the dry state.

Surprisingly, we have found that the tensile strength of such a dried expanded carrier material is only slightly influenced by the concentration of crosslinking agent. The stability is, rather, controlled by the cellular structure, particularly the thickness of the cell walls. This can be adjusted by varying the concentration of the protein solution to be foamed. Thus the force required to fracture a test sample (10×2.5×2 cm) increases by a factor of 4 when the protein concentration in the initial solution is raised from 10% to 18%.

In the wet state, however, crosslinking has a stabilizing effect, and biodegradation of the materials is slower.

The water-storing capacity of the resulting materials is substantially the same as that achieved in Example 2.

### 4. A Further Alternative Water-Storage Material According to Present Invention

A sponge cloth of regenerated cellulose (eg, a domestic cloth as sold by Freudenberg & Co KG under the trade name Vileda) is sprayed with a 5% strength collagen hydrolysate solution having an average \overline{M}_{w} of 5,000 dalton, treated with superabsorber (1.25 g/1.25 cm²), and dried. Here again, the water-storing capacity obtained is comparable to that of the materials of Examples 2 and 3.

1. A water-storage material, comprising a flat biodegradable carrier material and a particulate water absorber, the particles of said water absorber being fixed in position on the carrier material in an evenly distributed fashion.

2. The water-storage material as defined in claim 1, wherein said particles of said water absorber are fixed on said carrier material by means of a biodegradable, hydrophilic adhesive agent.

3. The water-storage material as defined in claim 2, wherein said adhesive agent is water-swellable.

4. The water-storage material as defined in claim 2, wherein said adhesive agent is water-soluble.

5. The water-storage material as defined in claim 2, wherein said adhesive agent is a polypeptide.

6. The water-storage material as defined in claim 5, wherein said polypeptide is a collagen hydrolysate.

7. The water-storage material as defined in claim 6, wherein said collagen hydrolysate has an average molecular weight \overline{M}_{w} of from 1,000 to 20,000 dalton, preferably from 3,000 to 15,000 dalton.

8. The water-storage material as defined in claim 1, wherein said carrier material comprises a polysaccharide, polypeptide, and/or PVA.

9. The water-storage material as defined in claim 8, wherein said carrier material is crosslinked.

10. The water-storage material as defined in claim 8, wherein said carrier material comprises a plasticizer.

11. The water-storage material as defined in claim 8, wherein said polypeptide is gelatin.

12. The water-storage material as defined in claim 1, wherein said carrier material is a flexible carrier material.

13. The water-storage material as defined in claim 1, wherein said carrier material is an expanded carrier material.

14. The water-storage material as defined in claim 1, wherein said carrier material has a non-woven structure.

15. The water-storage material as defined in claim 1, wherein said carrier material has a network structure.

16. The water-storage material as defined in claim 2, wherein said carrier material comprises said adhesive agent.

17. The water-storage material as defined in claim 16, wherein said adhesive agent is one which is capable of being activated by water.

18. The water-storage material as defined in claim 1, wherein a particulate fertilizer is additionally bonded to said carrier material in an evenly distributed fashion.

19. The water-storage material as defined in claim 1, wherein said water absorber material comprises a superabsorber material.

20. The water-storage material as defined in claim 16, wherein said superabsorber material is selected from the group comprising polyacrylic acids, polyacrylamines, polyvinyl amines, and derivatives thereof.

21. The water-storage material as defined in claim 1, wherein said material is suitable for breakthrough of roots.

22. The water-storage material as defined in claim 13, wherein said carrier has an open-cell pore structure.
